(19) United States
(12) Patent Application Publication
(10) Pub. No.: US 2022/0110853 A1
(43) Pub. Date: Apr. 14, 2022

(54) IMPROVEMENT IN OR RELATING TO ORGANIC COMPOUNDS

(71) Applicant: GIVAUDAN SA, Vernier (CH)

(72) Inventors: Elisabeth RINAUDO, Deuil la Barre (FR); Benoit RIFLADE, Bazas (FR); Mathieu ZONGO, Lausanne (CH)

(21) Appl. No.: 17/422,346

(22) PCT Filed: Dec. 18, 2019
(86) PCT No.: PCT/EP2019/085842

§ 371 Date: Jul. 12, 2021

(30) Foreign Application Priority Data
Feb. 4, 2019 (GB) 1901500.7

(51) Int. Cl. A61K 8/73; A61K 8/06; A61Q 15/00; A61K 8/81

(52) U.S. Cl. CPC A61K 8/732; A61K 8/731; A61K 8/06; A61K 2800/56; A61K 8/736; A61K 8/733; A61K 8/8135; A61Q 15/00

(57) ABSTRACT

Disclosed is an encapsulated composition comprising at least one perfume and/or cosmetic ingredient that is entrapped in a matrix. The matrix comprises a starch and a hemicellulose.

The present invention relates to an encapsulated composition, to a process for preparing an encapsulated composition, to a use of a hemicellulose for modifying the properties of a starch matrix and to a use of an encapsulated composition.

It is known to employ encapsulated perfume compositions in leave-on personal care products, both to fragrance the human or animal body and to counteract malodor. Leave-on products are adapted for topical application to hair or skin of a subject and left on the body for a prolonged period of time. Particularly important categories of leave-on products are deodorants and antiperspirants. Body odor is undesirable and may even be considered unhygienic and anti-social. Body odors emanate as a result of the action of micro-flora on human sweat. Regular bathing to remove sweat can address the build-up of body malodor, but it is not always practical or possible to bath or shower on a frequent basis. Accordingly, the application of deodorants and antiperspirants has become an important aspect of modern body care regimens.

Antiperspirants and deodorants present a particular challenge for formulators of perfume compositions. Consumers communicate intimately with these products from the moment of application and for many hours thereafter, until the next opportunity to bathe or shower presents itself. In order to control perfume release, encapsulated perfume compositions have been used in such products. For a desired release profile, the encapsulated perfume compositions should not be so fragile that the capsules break and discharge the perfume at the slightest frictional contact with the surface of skin, hair or fabric. On the other hand, because sweating results from physical activity, capsules should break and release perfume in response to frictional forces or exposure to moisture by sweat. Such an “on-demand” perfume release can provide an olfactive cue for consumers, indicating a product's efficacy, which in turn inspires consumer confidence in a product.

In the field of deodorants and antiperspirants, two types of encapsulated perfume compositions have generally been employed for commercial applications. On one hand, starch encapsulates have been used for their ability to release perfume when moistened. Compositions based on core-shell microcapsules, on the other hand, have been employed for their ability to fracture and deliver perfume in response to mechanical action. With both kinds of encapsulates, desired perfume release can be difficult to achieve over a whole use cycle. In case of core-shell microcapsules, an individual might sweat without physical activity, which can lead to insufficient masking of body malodor. Furthermore, core-shell microcapsules need strong friction to break. Starch encapsulates, on the other hand, can have the disadvantage that fragrance release is too slow in case of sudden physical activity. Moreover, day activity and movement without sweating will not provide any release from starch encapsulates.

EP 2 897 578 B1 and EP 2 897 579 B1 propose perfume compositions comprising a combination of core-shell microcapsules with a moisture-triggered perfume delivery system. Such compositions allow for fragrance release either through activation by mechanical action or by moisture, herein referred to as “dual activation”.

However, deodorants and antiperspirants comprising such combinations still suffer from drawbacks. A main disadvantage is that two different encapsulated perfume compositions need to be provided, which increases the price and complexity of the formulation. Furthermore, certain types of core-shell microcapsules are controversial due to the use of formaldehyde, isocyanates, acrylates or imines, although the amount of these materials in consumer products lies well below the threshold limits imposed.

It is therefore a problem underlying the present invention to overcome the above-mentioned drawbacks in the prior art. In particular, it is a problem underlying the present invention to provide an encapsulated perfume composition, which is susceptible to both mechanical and moisture activation. Such a composition should be facile in manufacture, cost efficient and sustainable.

These problems are solved by an encapsulated composition according to claim 1. The composition comprises at least one perfume and/or cosmetic ingredient that is entrapped in a matrix. The matrix comprises a starch and a hemicellulose.

In the context of the present invention, the expression “hemicellulose” is to be understood as a polysaccharide selected from the group consisting of glucans, in particular xyloglucans, mannans, in particular glucomannans, and xylans, in particular arabinoxylans and glucuronoxylans.

It has been found that addition of a hemicellulose to a starch matrix leads to a modification of the matrix, improving its perfume release properties under moisture and mechanical (e.g. friction) activation. The resulting perfume encapsulates are facile and cost-effective in manufacture. Furthermore, they are prepared of naturally-based polysaccharides, which are non-toxic and biodegradable. Such encapsulates therefore have an increased consumer-appeal.

The starch can be a water-soluble modified starch. Such a starch can be made from raw starch or pre-gelatinized starch. It can be derived from tubers, legumes, cereals and grains, for example corn starch, wheat starch, rice starch, waxy corn starch, oat starch, cassava starch, waxy barley starch, waxy rice starch, sweet rice starch, amioca starch, potato starch, tapioca starch and mixtures thereof.

The water-soluble modified starch can be selected from the group consisting of bleached starch, hydroxypropyl starch, hydroxypropyl distarch phosphate, hydroxypropyl distarch glycerol, acetylated distarch phosphate, starch acetate esterified with acetic anhydride, starch acetate esterified with vinyl acetate, acetylated distarch adipate, acetylated distarch glycerol, starch sodium octenyl succinate and mixtures thereof.

Water-soluble modified starches have emulsifying and emulsion-stabilizing capacity. They have the ability to entrap fragrance and/or cosmetic oil droplets in the form of oil-in-water emulsions due to the hydrophobic character of the starch modifying agent.

The modified starches as described herein above bring numerous advantages including high emulsification and encapsulation performance, low viscosity, even at high solids content, and excellent oxidation resistance to ensure good fragrance and/or cosmetic preservation and stabilization of sensitive ingredients.

The hemicellulose can be a xyloglucan. Xyloglucans are the most abundant hemicellulose in the primary walls of non-graminaceous plants, often comprising 20% of the dry mass of the wall. A xyloglucan has a backbone composed of 1,4-linked β-D-glucose residues. Up to 75% of the backbone residues are substituted at C6 with mono-, di-, or trisaccharide sidechains. Preferably, the hemicellulose is a xyloglucan obtainable from tamarind seeds, in particular obtained from tamarind seeds, also known as “tamarind kernel powder” or “tamarind gum”. In tamarind gum, the side chains consist of one or two α-D-xylopyranosyl units, optionally capped with β-D-galactopyranosyl, α-L-arabinofuranosyl or β-D-xylopyranosyl.

An encapsulated composition according to the present invention can additionally comprise a compound selected from the group consisting of maltodextrin, mannitol and mixtures thereof. Maltodextrin and mannitol both increase the glass transition temperature of the matrix. Furthermore, maltodextrin is a film forming agent.

In a preferred aspect of the present invention, the encapsulated composition additionally comprises mannitol. In such a composition, the proportion of mannitol can be 1 to 30 wt.-%, preferably 5 to 15 wt.-%, even more preferably 8 to 12 wt.-%.

An encapsulated composition according to the present invention can additionally comprise a flowing agent selected from the group consisting of silicon dioxide, sodium salts, calcium salts and zeolites. The silicon dioxide can be selected from precipitated, fumed and colloidal silica. The sodium salt can be sodium sulphate. The calcium salt can be calcium carbonate. The use of a flowing agent affords a flowable powder that is not susceptible to caking.

The at least one perfume ingredient can belong to different classes of organic compounds, as varied as alcohols, ketones, esters, ethers, acetates, terpene hydrocarbons, nitrogenous or sulphurous heterocyclic compounds and essential oils, which can be of natural or synthetic origin. Many of these perfume ingredients are listed in reference texts, such as S. Arctander, Perfume and Flavor Chemicals, 1994, Montclair, N.J., USA.

Preferably, the at least one perfume ingredient has a boiling point determined at the normal standard pressure of 1013.25 hPa of 275° C. or lower and an odor detection threshold of less than or equal to 50 parts per billion (ppb).

Preferably, at least 50 wt.-%, more particularly at least 60 wt.-%, and still more particularly at least 80 wt.-%, of the perfume ingredients have a c Log[P] of 2.0 or greater, and more particularly 2.5 or greater, still more particularly 3.0 or greater. The values of c Log[P] of fragrance ingredients have been reported in many databases, including the Pomona 92 database, available from Daylight Chemical Information Systems, Inc., Daylight CIS, Irvine, Calif.

The at least one perfume ingredient can be selected from the group consisting of ADOXAL™ (2,6,10-trimethylundec-9-enal), AGRUMEX™ (2-(tert-butyl)cyclohexyl acetate), ALDEHYDE C 10 DECYLIC (decanal), ALDEHYDE C 11 MOA (2-methyldecanal), ALDEHYDE C 11 UNDECYLENIC (undec-10-enal), ALDEHYDE C 11 UNDECYLIC (undecanal), ALDEHYDE C 12 LAURIC (dodecanal), ALDEHYDE C 12 MNA PURE (2-methylundecanal), ALDEHYDE ISO C 11 ((E)-undec-9-enal), ALDEHYDE MANDARINE 10%/TEC ((E)-dodec-2-enal), ALLYL AMYL GLYCOLATE (allyl 2-(isopentyloxy)acetate), ALLYL CYCLOHEXYL PROPIONATE (allyl 3-cyclohexylpropanoate), ALLYL OENANTHATE (allyl heptanoate), AMBER CORE™ (1-((2-(tert-butyl)cyclohexyl)oxy)butan-2-ol), AMBERMAX™ (1,3,4,5,6,7-hexahydro-.beta.,1,1,5,5-pentamethyl-2H-2,4a-methanonaphthal-ene-8-ethanol), AMYL SALICYLATE (pentyl 2-hydroxybenzoate), APHERMATE (1-(3,3-dimethylcyclohexyl)ethyl formate), BELAMBRE™ ((1R,2S,4R)-2′-isopropyl-1,7,7-trimethylspiro[bicyclo[2.2.1]heptane-2,4′-[1,3]dioxane]), BIGARYL (8-(sec-butyl)-5,6,7,8-tetrahydroquinoline), BOISAMBRENE™ FORTE™ ((ethoxymethoxy)cyclododecane), BOISIRIS™ ((1S,2R,5R)-2-ethoxy-2,6,6-trimethyl-9-methylenebicyclo[3.3.1]nonane), BORNYL ACETATE ((2S,4S)-1,7,7-trimethylbicyclo[2.2.1]heptan-2-yl acetate), BUTYL BUTYRO LACTATE (1-butoxy-1-oxopropan-2-yl butyrate), BUTYL CYCLOHEXYL ACETATE PARA (4-(tert-butyl)cyclohexyl acetate), CARYOPHYLLENE ((Z)-4,11,11-trimethyl-8-methylenebicyclo[7.2.0]undec-4-ene), CASHMERAN™ (1,1,2,3,3-pentamethyl-2,3,6,7-tetrahydro-1H-inden-4(5H)-one), CASSYRANE™ (5-tert-butyl-2-methyl-5-propyl-2H-furan), CITRAL ((E)-3,7-dimethylocta-2,6-dienal), CITRAL LEMAROME™ N ((E)-3,7-dimethylocta-2,6-dienal), CITRATHAL™ R ((Z)-1,1-diethoxy-3,7-dimethylocta-2,6-diene), CITRONELLAL (3,7-dimethyloct-6-enal), CITRONELLOL (3,7-dimethyloct-6-en-1-ol), CITRONELLYL ACETATE (3,7-dimethyloct-6-en-1-yl acetate), CITRONELLYL FORMATE (3,7-dimethyloct-6-en-1-yl formate), CITRONELLYL NITRILE (3,7-dimethyloct-6-enenitrile), CITRONELLYL PROPIONATE (3,7-dimethyloct-6-en-1-yl propionate), CLONAL (dodecanenitrile), CORANOL (4-cyclohexyl-2-methylbutan-2-ol), COSMONE™ ((Z)-3-methylcyclotetradec-5-enone), CYCLAMEN ALDEHYDE (3-(4-isopropylphenyl)-2-methylpropanal), CYCLOGALBANATE (allyl 2-(cyclohexyloxy)acetate), CYCLOHEXYL SALICYLATE (cyclohexyl 2-hydroxybenzoate), CYCLOMYRAL (8,8-dimethyl-1,2,3,4,5,6,7,8-octahydronaphthalene-2-carbaldehyde), DAMASCENONE ((E)-1-(2,6,6-trimethylcyclohexa-1,3-dien-1-yl)but-2-en-1-one), DAMASCONE ALPHA ((E)-1-(2,6,6-trimethylcyclohex-2-en-1-yl)but-2-en-1-one), DAMASCONE DELTA ((E)-1-(2,6,6-trimethylcyclohex-3-en-1-yl)but-2-en-1-one), DECENAL-4-TRANS ((E)-dec-4-enal), DELPHONE (2-pentylcyclopentanone), DIHYDRO ANETHOLE (propanedioic acid 1-(1-(3,3-dimethylcyclohexyl)ethyl) 3-ethyl ester), DIHYDRO JASMONE (3-methyl-2-pentylcyclopent-2-enone), DIMETHYL BENZYL CARBINOL (2-methyl-1-phenylpropan-2-ol), DIMETHYL BENZYL CARBINYL ACETATE (2-methyl-1-phenylpropan-2-yl acetate), DIMETHYL BENZYL CARBINYL BUTYRATE (2-methyl-1-phenylpropan-2-yl butyrate), DIMETHYL OCTENONE (4,7-dimethyloct-6-en-3-one), DIMETOL (2,6-dimethylheptan-2-ol), DIPENTENE (1-methyl-4-(prop-1-en-2-yl)cyclohex-1-ene), DUPICAL™ ((E)-4-((3aS,7aS)-hexahydro-1H-4,7-methanoinden-5(6H)-ylidene)butanal), EBANOL™ ((E)-3-methyl-5-(2,2,3-trimethylcyclopent-3-en-1-yl)pent-4-en-2-ol), ETHYL CAPROATE (ethyl hexanoate), ETHYL CAPRYLATE (ethyl octanoate), ETHYL LINALOOL ((E)-3,7-dimethylnona-1,6-dien-3-ol), ETHYL LINALYL ACETATE ((Z)-3,7-dimethylnona-1,6-dien-3-yl acetate), ETHYL OENANTHATE (ethyl heptanoate), ETHYL SAFRANATE (ethyl 2,6,6-trimethylcyclohexa-1,3-diene-1-carboxylate), EUCALYPTOL ((1s,4s)-1,3,3-trimethyl-2-oxabicyclo[2.2.2]octane), FENCHYL ACETATE ((2S)-1,3,3-trimethylbicyclo[2.2.1]heptan-2-yl acetate), FENCHYL ALCOHOL ((1S,2R,4R)-1,3,3-trimethylbicyclo[2.2.1]heptan-2-ol), FIXOLIDE™ (1-(3,5,5,6,8,8-hexamethyl-5,6,7,8-tetrahydronaphthalen-2-yl)ethanone), FLORALOZONE™ (3-(4-ethylphenyl)-2,2-dimethylpropanal), FLORHYDRAL (3-(3-isopropylphenyl)butanal), FLOROCYCLENE™ ((3aR,6S,7aS)-3a,4,5,6,7,7a-hexahydro-1H-4,7-methanoinden-6-yl propionate), FLOROPAL™ (2,4,6-trimethyl-4-phenyl-1,3-dioxane), FRESKOMENTHE™ (2-(sec-butyl)cyclohexanone), FRUITATE ((3aS,4S,7R,7aS)-ethyl octahydro-1H-4,7-methanoindene-3a-carboxylate), FRUTONILE (2-methyldecanenitrile), GALBANONE™ PURE (1-(3,3-dimethylcyclohex-1-en-1-yl)pent-4-en-1-one), GARDOCYCLENE™ ((3aR,6S,7aS)-3a,4,5,6,7,7a-hexahydro-1H-4,7-methanoinden-6-yl isobutyrate), GERANIOL ((E)-3,7-dimethylocta-2,6-dien-1-ol), GERANYL ACETATE SYNTHETIC ((E)-3,7-dimethylocta-2,6-dien-1-yl acetate), GERANYL ISOBUTYRATE ((E)-3,7-dimethylocta-2,6-dien-1-yl isobutyrate), GIVESCONE™ (ethyl 2-ethyl-6,6-dimethylcyclohex-2-enecarboxylate), HABANOLIDE™ ((E)-oxacyclohexadec-12-en-2-one), HEDIONE™ (methyl 3-oxo-2-pentylcyclopentaneacetate), HERBANATE™ ((2S)-ethyl 3-isopropylbicyclo[2.2.1]hept-5-ene-2-carboxylate), HEXENYL-3-CIS BUTYRATE ((Z)-hex-3-en-1-yl butyrate), HEXYL CINNAMIC ALDEHYDE ((E)-2-benzylideneoctanal), HEXYL ISOBUTYRATE (hexyl isobutyrate), HEXYL SALICYLATE (hexyl 2-hydroxybenzoate), INDOFLOR™ (4,4a,5,9b-tetrahydroindeno[1,2-d][1,3]dioxine), IONONE BETA ((E)-4-(2,6,6-trimethylcyclohex-1-en-1-yl)but-3-en-2-one), IRISONE ALPHA ((E)-4-(2,6,6-trimethylcyclohex-2-en-1-yl)but-3-en-2-one), IRONE ALPHA ((E)-4-(2,5,6,6-tetramethylcyclohex-2-en-1-yl)but-3-en-2-one), ISO E SUPER™ (1-(2,3,8,8-tetramethyl-1,2,3,4,5,6,7,8-octahydronaphthalen-2-yl)ethanone), ISOCYCLOCITRAL (2,4,6-trimethylcyclohex-3-enecarbaldehyde), ISONONYL ACETATE (3,5,5-trimethylhexyl acetate), ISOPROPYL METHYL-2-BUTYRATE (isopropyl 2-methyl butanoate), ISORALDEINE™ 70 ((E)-3-methyl-4-(2,6,6-trimethylcyclohex-2-en-1-yl)but-3-en-2-one), JASMACYCLENE™ ((3aR,6S,7aS)-3a,4,5,6,7,7a-hexahydro-1H-4,7-methanoinden-6-yl acetate), JASMONE CIS ((Z)-3-methyl-2-(pent-2-en-1-yl)cyclopent-2-enone), KARANAL™ (5-(sec-butyl)-2-(2,4-dimethylcyclohex-3-en-1-yl)-5-methyl-1,3-dioxane), KOAVONE ((Z)-3,4,5,6,6-pentamethylhept-3-en-2-one), LEAF ACETAL ((Z)-1-(1-ethoxyethoxy)hex-3-ene), LEMONILE™ ((2E,6Z)-3,7-dimethylnona-2,6-dienenitrile), LIFFAROME™ GIV ((Z)-hex-3-en-1-yl methyl carbonate), LILIAL™ (3-(4-(tert-butyl)phenyl)-2-methylpropanal), LINALOOL (3,7-dimethylocta-1,6-dien-3-ol), LINALYL ACETATE (3,7-dimethylocta-1,6-dien-3-yl acetate), MAHONIAL™ ((4E)-9-hydroxy-5,9-dimethyl-4-decenal), MALTYL ISOBUTYRATE (2-methyl-4-oxo-4H-pyran-3-yl isobutyrate), MANZANATE (ethyl 2-methylpentanoate), MELONAL™ (2,6-dimethylhept-5-enal), MENTHOL (2-isopropyl-5-methylcyclohexanol), MENTHONE (2-isopropyl-5-methylcyclohexanone), METHYL CEDRYL KETONE (1-((1S,8aS)-1,4,4,6-tetramethyl-2,3,3a,4,5,8-hexahydro-1H-5,8a-methanoazulen-7-yl)ethanone), METHYL NONYL KETONE EXTRA (undecan-2-one), METHYL OCTYNE CARBONATE (methyl non-2-ynoate), METHYL PAMPLEMOUSSE (6,6-dimethoxy-2,5,5-trimethylhex-2-ene), MYRALDENE (4-(4-methylpent-3-en-1-yl)cyclohex-3-enecarbaldehyde), NECTARYL (2-(2-(4-methylcyclohex-3-en-1-yl)propyl)cyclopentanone), NEOBERGAMATE^{T}M FORTE (2-methyl-6-methyleneoct-7-en-2-yl acetate), NEOFOLIONE™ ((E)-methyl non-2-enoate), NEROLIDYLE™ ((Z)-3,7,11-trimethyldodeca-1,6,10-trien-3-yl acetate), NERYL ACETATE HC ((Z)-3,7-dimethylocta-2,6-dien-1-yl acetate), NONADYL (6,8-dimethylnonan-2-ol), NONENAL-6-CIS ((Z)-non-6-enal), NYMPHEAL™ (3-(4-isobutyl-2-methylphenyl)propanal), ORIVONE™ (4-(tert-pentyl)cyclohexanone), PARADISAMIDE™ (2-ethyl-N-methyl-N-(m-tolyl)butanamide), PELARGENE (2-methyl-4-methylene-6-phenyltetrahydro-2H-pyran), PEONILE™ (2-cyclohexylidene-2-phenylacetonitrile), PETALIA™ (2-cyclohexylidene-2-(o-tolyl)acetonitrile), PIVAROSE™ (2,2-dimethyl-2-pheylethyl propanoate), PRECYCLEMONE™ B (1-methyl-4-(4-methylpent-3-en-1-yl)cyclohex-3-enecarbaldehyde), PYRALONE™ (6-(sec-butyl)quinoline), RADJANOL™ SUPER ((E)-2-ethyl-4-(2,2,3-trimethylcyclopent-3-en-1-yl)but-2-en-1-ol), RASPBERRY KETONE (N112) (4-(4-hydroxyphenyl)butan-2-one), RHUBAFURANE™ (2,2,5-trimethyl-5-pentylcyclopentanone), ROSACETOL (2,2,2-trichloro-1-phenylethyl acetate), ROSALVA (dec-9-en-1-ol), ROSYFOLIA ((1-methyl-2-(5-methylhex-4-en-2-yl)cyclopropyl)-methanol), ROSYRANE™ SUPER (4-methylene-2-phenyltetrahydro-2H-pyran), SERENOLIDE (2-(1-(3,3-dimethylcyclohexyl)ethoxy)-2-methylpropyl cyclopropanecarboxylate), SILVIAL™ (3-(4-isobutylphenyl)-2-methylpropanal), SPIROGALBANONE™ (1-(spiro[4.5]dec-6-en-7-yl)pent-4-en-1-one), STEMONE™ ((E)-5-methylheptan-3-one oxime), SUPER MUGUET™ ((E)-6-ethyl-3-methyloct-6-en-1-ol), SYLKOLIDE™ ((E)-2-((3,5-dimethylhex-3-en-2-yl)oxy)-2-methylpropyl cyclopropanecarboxylate), TERPINENE GAMMA (1-methyl-4-propan-2-ylcyclohexa-1,4-diene), TERPINOLENE (1-methyl-4-(propan-2-ylidene)cyclohex-1-ene), TERPINYL ACETATE (2-(4-methylcyclohex-3-en-1-yl)propan-2-yl acetate), TETRAHYDRO LINALOOL (3,7-dimethyloctan-3-ol), TETRAHYDRO MYRCENOL (2,6-dimethyloctan-2-ol), THIBETOLIDE (oxacyclohexadecan-2-one), TRIDECENE-2-NITRILE ((E)-tridec-2-enenitrile), UNDECAVERTOL ((E)-4-methyldec-3-en-5-ol), VELOUTONE™ (2,2,5-trimethyl-5-pentylcyclopentanone), VIRIDINE™ ((2,2-dimethoxyethyl)benzene), ZINARINE™ (2-(2,4-dimethylcyclohexyl)pyridine) and mixtures thereof.

In context of the present invention, cosmetic ingredients may be selected from the group consisting of emollients, smoothening actives, hydrating actives, soothing and relaxing actives, decorative actives, anti-aging actives, draining actives, remodeling actives, skin levelling actives, preservatives, anti-oxidant actives, antibacterial or bacteriostatic actives, cleansing actives, lubricating actives, structuring actives, hair conditioning actives, whitening actives, texturing actives, softening actives, anti-dandruff actives, and exfoliating actives.

Particularly useful cosmetic ingredients include hydrophobic polymers, such as alkyldimethylsiloxanes, polymethylsilsesquioxanes, polyethylene, polyisobutylene, styrene-ethylene-styrene and styrene-butylene-styrene block copolymers, mineral oils, such as hydrogenated isoparaffins, silicone oils, vegetable oils, such as argan oil, jojoba oil, aloe vera oil, fatty acids and fatty alcohols and their esters, glycolipides, phospholipides, sphingolipides, such as ceramides, sterols and steroids, terpenes, sesquiterpenes, triterpenes and derivatives thereof, essential oils, such as arnica oil, artemisia oil, bark tree oil, birch leaf oil, calendula oil, cinnamon oil, echinacea oil, eucalyptus oil, ginseng oil, jujube oil, helianthus oil, jasmine oil, lavender oil, lotus seed oil, perilla oil, rosmary oil, sandal wood oil, tea tree oil, thyme oil, valerian oil, wormwood oil, ylang ylang oil, yucca oil.

In an encapsulated composition according to the present invention, the proportion of the at least one perfume and/or cosmetic ingredient can be 10 to 50 wt.-%, preferably 20 to 47.5 wt.-%, even more preferably 30 to 45 wt.-%.

In an encapsulated composition according to the present invention, the proportion of starch can be 30 to 90 wt.-%, preferably 35 to 80 wt.-%, even more preferably 40 to 70 wt.-%.

In an encapsulated composition according to the present invention, the proportion of hemicellulose can be 0.02 to 20 wt.-%, preferably 0.1 to 10 wt.-%, even more preferably 0.5 to 5 wt.-%. In a particularly preferred embodiment of the present invention, the proportion of hemicellulose is 0.75 to 2 wt.-%.

An encapsulated composition according to the present invention can be in particulate form. More specifically, 90 vol.-% of the particles can have a size from 1 to 1000 μm, preferably from 2 to 400 μm, even more preferably from 3 to 200 μm. The volume median particle size D(50) can be 5 to 200 μm, preferably 10 to 120 μm, even more preferably 20 to 100 μm.

In present context, the size of the particles is measured by laser scattering using a Beckman Coulter LS13 320 particle size analyser. This device measures the size distribution of the particles in dry powder form by using the principles of light scattering. In a standard operation procedure, the particle diameter is expressed by volume and/or by number.

A further aspect of the present invention relates to a process for preparing an encapsulated composition, in particular a composition as described herein above. The process comprises the steps of:
a) Preparing an emulsion comprising at least one perfume and/or cosmetic ingredient, a starch and a hemicellulose in water;
b) Subjecting the emulsion to drying, in particular spray-drying or adsorption onto silicon dioxide, to obtain an encapsulated composition.

Preferably, the emulsion prepared in step a) additionally comprises a compound selected from the group consisting of maltodextrin, mannitol and mixtures thereof. For preparing the emulsion, the starch, the hemicellulose, and optionally also the maltodextrin and/or mannitol, are typically added to water under stirring. Thereafter, the at least one perfume and/or cosmetic ingredient is normally added under continued agitation. But a different order of addition is also possible. High-shear homogenization can then be used in order to obtain an emulsion with a desired droplet size.

In step b), spray-drying can be used to obtain an encapsulated composition. Spray drying techniques are well known in the art. The emulsion is usually pumped into a spray drying apparatus and atomized through a nozzle or spinning disk into a drying chamber. The emulsion may be entrained in a fluid (such as air) that moves inside the drying chamber. The fluid, which may be heated, causes the water to evaporate, leaving behind the encapsulated composition, which can then be collected at the dryer outlet. A typical inlet temperature for spray drying might be set at around 200° C., and an outlet temperature of around 100° C.

In a process as described hereinabove, the emulsion prepared in step a) can have a proportion of the at least one perfume and/or cosmetic ingredient of 2 to 60 wt.-%, preferably 5 to 40 wt.-%, even more preferably 10 to 30 wt.-%.

In a process as described hereinabove, the emulsion prepared in step a) can have a proportion of starch of 2 to 60 wt.-%, preferably 5 to 40 wt.-%, even more preferably 10 to 30 wt.-%.

In a process as described hereinabove, the emulsion prepared in step a) can have a proportion of hemicellulose of 0.001 to 10 wt.-%, preferably 0.01 to 5 wt.-%, even more preferably 0.1 to 1.0 wt.-%.

In a process as described hereinabove, the emulsion prepared in step a) can have a proportion of mannitol of 0.5 to 15 wt.-%, preferably 1 to 10 wt.-%, even more preferably 2 to 8 wt.-%.

In a process as described hereinabove, the emulsion prepared in step a) can have a proportion of water of 10 to 90 wt.-%, preferably 30 to 80 wt.-%, even more preferably 50 to 60 wt.-%.

When the proportion of the at least one perfume and/or cosmetic ingredient, the proportion of starch and the proportion of hemicellulose is low, and the proportion of water is high, the energy cost to the process is high because of the need to remove high levels of water. The limiting factor on the proportion of starch is the need to be able to process the mixture. Higher levels of starch can be accommodated as long as the mixture can still be subjected to spray-drying. Other additives may be incorporated to reduce viscosity of the starch/water mixture and improve ease of handling. Suitable examples include emulsifiers and plasticizers.

In order to avoid any ambiguity, if applicable, the optional features stated hereinabove in connection with the encapsulated composition can also be realized in the present process.

The present invention also relates to an encapsulated composition obtainable by the process as described hereinabove.

A further aspect of the present invention relates to a use of a hemicellulose for modifying the properties of a starch matrix, in particular of an encapsulated composition comprising at least one perfume and/or cosmetic ingredient that is entrapped in the starch matrix, more specifically a composition as described herein above. The present invention also relates to a method for modifying the properties of a starch matrix, in particular of an encapsulated composition comprising at least one perfume and/or cosmetic ingredient that is entrapped in the starch matrix, more specifically a composition as described herein above, by adding a hemicellulose when the matrix is formed, in particular in a process as described herein above.

The present invention also relates a consumer product, preferably a personal care product, even more preferably a deodorant or antiperspirant, comprising an encapsulated composition as described herein above. As used herein, a “consumer product” means an article intended to be used or consumed in the form in which it is sold, and not intended for subsequent commercial manufacture or modification. The amount of encapsulated composition in such a product can be 0.02 to 20 wt.-%, preferably 0.1 to 10 wt.-%, even more preferably 0.5 to 5 wt.-%.

A further aspect of the present invention relates to a use of an encapsulated composition as described herein above for reducing the amount of at least one perfume and/or cosmetic ingredient in a personal care product, in particular a deodorant or antiperspirant. The present invention also relates to a method for reducing the amount of at least one perfume and/or cosmetic ingredient in a personal care product, in particular a deodorant or antiperspirant, by encapsulating the at least one perfume and/or cosmetic ingredient in an encapsulated composition as described herein above.

Further features and particular advantages of the present invention become apparent from the following description of several examples.

### EXAMPLE 1—ADDITIVE SCREENING

For additive screening, several encapsulated perfume compositions comprising a starch matrix with 1 wt.-% of additive (chitosan, alginate, polyvinylalcohol or tamarind kernel powder) were prepared by the method according to Example 2.

For olfactive assessment, 0.4 wt.-% of encapsulated perfume composition was added in each case to a standard antiperspirant base. 0.2 g of product was placed in the middle of a fabric patch and the product was spread on the fabric by means of a spatula. The product was left on the fabric to dry for 4 h. The following assessments were then conducted by a panel of 20 panelists by attributing a score following a scale 0 to 5 for each stage:
Pre-Activation: Dry assessment 4 h after application;
Friction-Activation: Fold the fabric and rub it twice; Assess boost immediately after activation;
Moisture-Activation: Spray water twice directly on the fabric; Assess boost immediately after activation.

The values for friction-activation represent scores obtained in addition to pre-activation, those for moisture-activation represent scores obtained in addition to friction-activation.

Table 1 summarizes the results of the additive screening. Entry 1 corresponds to the benchmark, for which conventional starch encapsulation with no additive was used. Entries 2, 3 and 4 relate to starch matrices that were modified in each case with 1 wt.-% of either chitosan, alginate or polyvinylalcohol. In all three of these examples, increased pre-activation intensities with decreased friction- and water-activation intensities were observed. Addition of 1 wt.-% of tamarind kernel powder (entry 5) also showed slightly increased pre-activation intensity. The friction activation, on the other hand, was significantly increased with respect to the benchmark. Finally, no significant increase in moisture activation was observed in this entry.

TABLE 1

| | | Pre- | Friction- | Moisture- |
|---|---|---|---|---|
| Entry | Additive | Activation | Activation | Activation |
| 1 | none | 1.3 | 1.7 | 4.0 |
| 2 | Chitosan | 2.3 | 0.2 | 2.6 |
| 3 | Alginate | 1.8 | 0.2 | 2.6 |
| 4 | Polyvinylalcohol | 1.5 | 0.3 | 2.6 |
| 5 | Tamarind kernel | 1.8 | 2.7 | 4.3 |
| | powder | | | |

### EXAMPLE 2—PREPARATION OF ENCAPSULATED COMPOSITIONS

Tap water (55.0 g) was weighted into a stainless steel beaker. Starch sodium octenyl succinate E1450 (18.7 g), starch modified Hi-Cap 100 (2.2 g), maltodextrin Glucidex IT-19 (5.3 g) and the additive as stated in Example 1 (0.5 g) were subsequently weighted into the same beaker. The resulting mixture was first manually stirred with a stainless steel rod and then homogenized with an IKA T25 Ultra-Turrax Homogenizer at 13,500 rpm to obtain a homogeneous solution. To this resulting mixture, perfume oil (17.8 g) was added. High shear mixing was then carried out for 20-30 min at 22,000-24,000 rpm using the same Homogenizer to produce an emulsion. The droplet size was controlled by dynamic light scattering to be between 0.5 and 2 μm.

The emulsion was subjected to spray drying using a LabPlant SD-06 Spray Dryer. The spray drying process parameters were as follows:
Inlet Temperature: 190° C.
Outlet Temperature: 90° C.
Peristaltic pump speed: 485 mL/h
Air flow rate: 3.7 m/s

The resulting spray dried powder was mixed with silicon dioxide Aerosil 200 (0.5 g) in a closed mixing vessel.

### EXAMPLE 3—PREPARATION OF ALTERNATIVE ENCAPSULATED COMPOSITION ACCORDING TO THE PRESENT INVENTION

Tap water (45.0 g) was weighted into a stainless steel beaker. Starch sodium octenyl succinate E1450 (21.9 g), mannitol 60 (5.5 g) and tamarind kernel powder (0.5 g) were subsequently weighted into the same beaker. The resulting mixture was first manually stirred with a stainless steel rod and then homogenized with an IKA T25 Ultra-Turrax Homogenizer at 13,500 rpm to obtain a homogeneous solution. To the resulting mixture, perfume oil (27.3 g) was added. High shear mixing was then carried out for 20-30 min at 22,000-24,000 rpm using the same Homogenizer to produce an emulsion. The droplet size was controlled by dynamic light scattering to be between 0.5 and 2 μm.

The emulsion was subjected to spray drying using a LabPlant SD-06 Spray Dryer. The spray drying process parameters were as follows:
Inlet Temperature: 190° C.
Outlet Temperature: 90° C.
Peristaltic pump speed: 485 mL/h
Air flow rate: 3.7 m/s

The resulting spray dried powder was mixed with silicon dioxide Aerosil 200 (0.5 g) in a closed mixing vessel.

### EXAMPLE 4—STABILITY OF ENCAPSULATED COMPOSITION ACCORDING TO THE PRESENT INVENTION

The stability of an encapsulated composition according to the present invention, prepared according to Example 2 with tamarind kernel powder as an additive, was assessed and compared with a conventional starch encapsulate containing the same fragrance. The same olfactive assessment protocol as in Example 1 was used. Table 2 summarizes the results after aging the samples at 37° C. and 70% relative humidity for 1 month. It can be seen that the superior olfactive performance of the encapsulated composition according to the present invention (entries 1 and 2) compared to a conventional starch encapsulate (entries 3 and 4) is retained at all stages (pre-activation, friction-activation and moisture-activation).

TABLE 2

| | | | Pre- | Friction- | Moisture- |
|---|---|---|---|---|---|
| Entry | Sample | | Activation | Activation | Activation |
| 1 | Encapsulated | Fresh sample | 2.5 | 3.3 | 3.8 |
| 2 | composition | After 1 month | 2.0 | 2.8 | 3.0 |
| | according to | aged at 37° C. | | | |
| | present | | | | |
| | invention | | | | |
| 3 | Conventional | Fresh sample | 2.0 | 2.8 | 3.3 |
| 4 | starch | After 1 month | 1.5 | 2.3 | 2.5 |
| | encapsulate | aged at 37° C. | | | |

### EXAMPLE 5—STABILITY OF ALTERNATIVE ENCAPSULATED COMPOSITION ACCORDING TO THE PRESENT INVENTION

The stability of an alternative encapsulated composition according to the present invention, prepared according to Example 3, was assessed. The same olfactive assessment protocol as in Example 1 was used. Table 3 summarizes the results for a fresh sample as well as after aging samples at 4° C. or 37° C., respectively, and 70% relative humidity for 1 month. It can be seen that particularly low pre-activation intensity was achieved, in particular after 1 month of storage at 37° C. Furthermore, good olfactive performance was observed upon friction-activation and moisture-activation, even after storage under challenging conditions.

TABLE 3

| | | | Pre- | Friction- | Moisture- |
|---|---|---|---|---|---|
| Entry | Sample | | Activation | Activation | Activation |
| 1 | Encapsulated | Fresh sample | 1.5 | 3.0 | 3.5 |
| 2 | composition | After 1 month | 1.5 | 3.0 | 3.0 |
| | according to | aged at 4° C. | | | |
| 3 | present | After 1 month | 1.0 | 2.5 | 2.5 |
| | invention | aged at 37° C. | | | |

1. An encapsulated composition comprising at least one perfume and/or cosmetic ingredient that is entrapped in a matrix, wherein the matrix comprises a starch and a hemicellulose.

2. The encapsulated composition according to claim 1, wherein the starch is a water-soluble modified starch.

3. The encapsulated composition according to claim 2, wherein the water-soluble modified starch is selected from the group consisting of bleached starch, hydroxypropyl starch, hydroxypropyl distarch phosphate, hydroxypropyl distarch glycerol, acetylated distarch phosphate, starch acetate esterified with acetic anhydride, starch acetate esterified with vinyl acetate, acetylated distarch adipate, acetylated distarch glycerol, starch sodium octenyl succinate and mixtures thereof.

4. The encapsulated composition according to claim 1, wherein the hemicellulose is a xyloglucan, in particular a xyloglucan obtainable from tamarind seeds.

5. The encapsulated composition according to claim 1, additionally comprising a compound selected from the group consisting of maltodextrin, mannitol and mixtures thereof.

6. The encapsulated composition according to claim 1, additionally comprising a flowing agent selected from the group consisting of silicon dioxide, sodium salts, calcium salts and zeolites.

7. The encapsulated composition according to claim 1, wherein the proportion of the at least one perfume and/or cosmetic ingredient is 10 to 50 wt.-%.

8. The encapsulated composition according to claim 1, wherein the proportion of starch is 30 to 90 wt.-%.

9. The encapsulated composition according to claim 1, wherein the proportion of hemicellulose is 0.02 to 20 wt.-%.

10. The encapsulated composition according to claim 1, wherein the composition is in particulate form.

11. A process for preparing an encapsulated composition, the process comprising the steps of:
a) Preparing an emulsion comprising at least one perfume and/or cosmetic ingredient, a starch and a hemicellulose in water;
b) Subjecting the emulsion to drying, in particular spray-drying or adsorption onto silicon dioxide, to obtain an encapsulated composition.

12. (canceled)

13. (canceled)

14. A consumer product comprising an encapsulated composition according to claim 1.

15. (canceled)

16. The encapsulated composition according to claim 5, additionally comprising a flowing agent selected from the group consisting of silicon dioxide, sodium salts, calcium salts and zeolites.
